(19) United States
(12) Patent Application Publication
(10) Pub. No.: US 2021/0252078 A1
(43) Pub. Date: Aug. 19, 2021

(54) SPORE-BASED PROBIOTIC SUPPLEMENTATION IN CHICKENS AND EFFECT ON SALMONELLA LOADS

(71) Applicant: Microbiome Labs, LLC, Saint Augustine, FL (US)

(72) Inventors: Kiran Krishnan, Saint Augustine, FL (US); Dale M. Kriz, Saint Augustine, FL (US); Thomas F. Bayne, Saint Augustine, FL (US)

(73) Assignee: Microbiome Labs, LLC, Saint Augustine, FL (US)

(21) Appl. No.: 17/022,805

(22) Filed: Sep. 16, 2020

Related U.S. Application Data
(60) Provisional application No. 62/900,893, filed on Sep. 16, 2019.

(51) Int. Cl. A61K 35/742; A23K 50/75; A23K 10/16

(52) U.S. Cl. CPC A61K 35/742; A23K 10/16; A23K 50/75

(57) ABSTRACT

This disclosure relates to a method of evaluating effects of probiotic supplementation on performance and Salmonella load in broilers challenged with Salmonella enterica serovar Enteritidis, said probiotic containing Bacillus subtilis, Bacillus licheniformis, and a cellulose carrier.

This application claims the benefit of U.S. Provisional application No. 62/900,893, filed on Sep. 16, 2019, which is hereby incorporated by reference herein in its entirety.

### SEQUENCE LISTING

The Sequence Listing is submitted as an ASCII text file in the form of a file named 17022805_SEQUENCE_LISTING_FILE_ST25.txt (725 bytes) created on Oct. 26, 2020, which is herein incorporated by reference in its entirety.

### FIELD OF THE INVENTION

The present invention relates to a method of evaluating effects of probiotic supplementation on performance and Salmonella load in broilers challenged with Salmonella enterica serovar Enteritidis, said probiotic containing Bacillus subtilis, Bacillus licheniformis, and cellulose carrier.

### BACKGROUND

Interactions between the gut microbiota and host play an important role in the regulation of a multitude of physiological processes in many species, including domestic mammals and fowls.

Probiotics consist of live microorganisms that employ benefits to their host primarily by supporting the proliferation of beneficial gut microflora. Furthermore, probiotics modulate the frequency (i.e., population or density) of the tight junction proteins that act as a barrier in the intestinal paracellular pathway. By enhancing intestinal barrier function, probiotics serve as preventative agents to defend against adverse effects of pathogens, promoting positive effects on digestion and immune health. Additionally, it appears that the beneficial effects of probiotics may be strain-specific, with a majority of probiotic studies investigating Bifidobacterium and Lactobacillus strains in various special groups (i.e. diabetic, obese) of the general human population.

Accordingly, there exist a need for a method of administering Bacillus subtilis probiotic supplementation (B. subtilis composition) to animal subjects such as domestic birds or fowls.

### SUMMARY

The present invention relates to a method of administration of probiotic supplements or treating caecal Salmonella load, and Salmonella contamination in meat of chicken broilers using a probiotic containing Bacillus subtilis and Bacillus licheniformis. The probiotic supplement can optionally contain one or more additional components such as cellulose carrier.

### BRIEF DESCRIPTION OF THE DRAWINGS

FIG. 1 depicts, in one embodiment, the effects of probiotic supplementation with HU58 and Prepro on body weight gain. Data were gathered on days 7, 14, 21, 28, 35 and 42. The bars left to right refer to Control, Salmonella challenge, probiotic in feed, and probiotic in feed plus Salmonella challenge, per the days listed above.

FIG. 2 depicts, in one embodiment, the effects of probiotic supplementation with HU58 and Prepro on feed consumption. Data were gathered on days 7, 14, 21, 28, 35 and 42. The bars left to right refer to Control, Salmonella challenge, probiotic in feed, and probiotic in feed plus Salmonella challenge, per the days listed above.

FIG. 3 depicts, in one embodiment, the effects of probiotic supplementation with HU58 and Prepro on feed efficiency. Data were gathered on days 7, 14, 21, 28, 35 and 42. The bars left to right refer to Control, Salmonella challenge, probiotic in feed, and probiotic in feed plus Salmonella challenge, per the days listed above.

FIG. 4 depicts, in one embodiment, effects of probiotic supplementation with HU58 and Prepro on Caecal Salmonella Colonization by measuring Salmonella genomic DNA using rtPCR method. The copy numbers of S. enteritidis was expressed in log 10 CFU/g of caecal contents. Data were gathered on days 0, 5, 12 and 21. The bars left to right refer to Control, Salmonella challenge, probiotic in feed, and probiotic in feed plus Salmonella challenge, per the days listed above. Note: the negative numbers should in theory be “Non detectable”.

FIG. 5 depicts, in one embodiment, effects of probiotic supplementation with HU58 and Prepro on Caecal Campylobacter Colonization by measuring Campylobacter genomic DNA using rtPCR method. The copy numbers was expressed in log 10 CFU/g of caecal contents. Data were gathered on days 0, 5, 12 and 21. The bars left to right refer to Control, Salmonella challenge, probiotic in feed, and probiotic in feed plus Salmonella challenge, per the days listed above. Note: the negative numbers should in theory be “Non detectable”. Hence all the data here is “ND”.

FIG. 6 depicts, in one embodiment, effects of probiotic supplementation with HU58 and Prepro on bile anti-Salmonella specific IgA titers. Data were gathered on days 0, 5, 12 and 21. The bars left to right refer to Control, Salmonella challenge, probiotic in feed, and probiotic in feed plus Salmonella challenge, per the days listed above.

FIG. 7 depicts, in one embodiment, effects of probiotic supplementation with HU58 and Prepro on jejunal villi height and crypt depth (both in microns), and villi height:crypt depth ratio. The left data panels show bars of villi ht. (left) and crypt depth (right), top panel day 21, bottom panel day 42; while the right data panels show only and villi height:crypt depth ratio, top panel day 21, bottom panel day 42.

### DETAILED DESCRIPTION

In its principal embodiment, a Bacillus subtilis (B. subtilis) containing composition is used for probiotic supplementation in an avian chicken subject. HU58 (“ProBiotene™”) is a strain of Bacillus subtilis, a preparation of which is manufactured by Viridis BioPharma Pvt. Ltd., Mumbai, India. Bacillus subtilis HU58 has been deposited with the National Center for Biotechnology Research under the accession number EF101709. The Bacillus Genetic Stock Center (“BGSC”) assigned number for Bacillus HU58 is 3A34, and the NCIMB Ltd. assigned strain number is 30283.

A strain of Bacillus licheniformis is used in the probiotic formulation. SL-307 is a strain of Bacillus licheniformis used in the probiotic formulation, a preparation of which was manufactured by Synergia Life Sciences Pvt. Ltd., Mumbai, India.

“Prepro” is Bacillus licheniformis and a cellulose carrier. Prepro is available from Microbiome Labs (Glenview, Ill., USA).

In an embodiment, the probiotic composition includes 10 mg/Kg feed of HU58+100 mg/kg feed of Prepro.

In one example, a study describing the effects of HU58+Prepro probiotic supplementation on performance, caecal Salmonella load, and Salmonella contamination in meat of broilers challenged with Salmonella enterica serovar Enteritidis is attached as an appendix hereto. The probiotic supplement can optionally contain one or more additional components such as cellulose carrier.

The effect of probiotic supplementation on caecal Campylobacter load post Salmonella infection in broiler birds is also determined.

The invention contemplates poultry birds and other fowls.

This invention is further illustrated by the following additional examples that should be construed as limiting. Those of skill in the art should, in light of the present disclosure, appreciate that many changes can be made to the specific embodiments that are disclosed and still obtain a like or similar result without departing from the spirit and scope of the invention.

### EXAMPLES

Evaluation of the effects of Probiotic supplementation on performance and Salmonella load in broilers challenged with Salmonella enterica serovar Enteritidis.

Objective: The overall objective is to determine the effects of probiotic supplementation (B. subtilis HU58+Prepro) on performance, caecal Salmonella load, and Salmonella contamination in meat of broilers challenged with Salmonella enterica serovar Enteritidis.

Study Design

A total of 360 one-day-old broiler chicks were randomly distributed to four experimental groups. Each treatment will be replicated in 6 pens (n=6) with 15 chicks per pen.

The five experimental groups were:

1. Control

2. Control+Challenge

3. Probiotics in feed

4. Probiotics in feed+Challenge

In this embodiment the probiotics consisted of 10 mg/Kg feed of HU58+100 mg/kg feed of Prepro. Probiotics were mixed with feed. Each treatment was replicated in 6 pens (n=6) with 15 chicks per pen. The four experimental groups were were fed a basal diet that meet the minimal NRC requirement. The basal diet was based on corn and soybean diet. Body weight and feed consumption was measured at weekly intervals and body weight gain and feed efficiency was calculated. Average body weight of birds in each pen was measured weekly.

At 21 d of age, all birds in experimental groups were inoculated orally with 250 μl of 1×10⁹ CFU S. enteritidis.

At 42 d of age, all birds were euthanized.

Sample Collection

Samples were collected from one bird per replication (6 birds per treatment) at d 0, 5, 12, and 21 d of age. Jejunal section was collected in formalin to study the villi height and crypt depth parameters. Caecal content was frozen for analyzing the total Salmonella and Campylobacter loads. Bile and serum was analyzed for anti-Salmonella IgA.

Effect of probiotic supplementation on caecal Salmonella enterica ser. enteritidis load post Salmonella infection in broiler birds.

At 0, 5, 12, and 21 d post infection, caecal contents were collected and analyzed for S. enteritidis load by real time PCR. 0, 5, 12, and 21 d post infection, caecal samples were collected from one pen in each of the six replication pens (n=6). Bacterial genomic DNA was isolated as follows. Caecal samples (200 mg) were washed two times with 1× PBS. The cell pellet was resuspended in EDTA and treated with 20 mg/ml lysozyme for 30 min at 37° C., followed by treatment with lysis buffer containing 20% SDS and 0.1 mg/ml proteinase K (Sigma Aldrich, St Louis, Mo.) for 5 min at 80° C. The samples were incubated with 5 μL of RNase at 37° C. for 30 min. The cell lysate was incubated with 6M sodium chloride on ice for 10 min. The supernatant was collected after centrifugation at 400×g for 10 min. The DNA in the supernatant was precipitated with isopropanol and washed once in ice-cold ethanol. The DNA pellet was resuspended in TE buffer (10 mM Tris-HCl, 1 mM EDTA, pH 8.0) and stored at −20° C. until further use.

The DNA extracted from the different treatment groups was analyzed for S. enteritidis load by real-time PCR as described earlier using primers (F-GCAGCGGTTACTATTGCAGC (SEQ ID No. 1) and R-CTGTGACAGGGACATTTAGCG (SEQ ID No. 2)). The threshold cycle (Cq) values were determined by iQ5 software (Bio-Rad, Hercules, Calif.) when the fluorescence rises exponentially 2-fold above background. The copy numbers of S. enteritidis was expressed in log units (log 10 CFU/g digesta) as described earlier (Shanmugasundaram, et al., Poultry Science (2019) 98 (11): 5840-5846).

Effect of probiotic supplementation on caecal Campylobacter load post Salmonella infection in broiler birds.

At 0, 5, 12, and 21 d post infection, caecal contents were collected and analyzed for Campylobacter jejuni primers using known primers, as described above.

Effect of Probiotic Supplementation on Bile and Serum anti-Salmonella IgA Content post Salmonella infection in broiler birds.

Bile and serum samples were collected from one bird per pen at 0, 5, 12, and 21 d post infection and analyzed for anti-Salmonella IgA content using an enzyme-linked immunosorbent assay (ELISA). The primary and secondary antibody concentrations were established using checkerboard titrations with dilutions of bile and antigens. Salmonella antigen for coating was made by 3 consecutive freeze thaw cycles of pure culture of CP followed by mechanical lysing. The pure culture was lysed two times by glass beads size 425-600 μm (Sigma, St. Louis, Mo.) in a TissueLyser LT (Qiagen, Germany) for 5 minutes at 50 Hz. The lysed cells were centrifuged at 10,000×g for 10 min and the resultant supernatant was collected and stored at −70° C. until use. Flat-bottomed 96-well microtitration plates (Microlon 600® High Binding, Greiner, N.C., USA) were coated with 100 μl of 10 μg/ml of the antigen diluted in 0.1M carbonate buffer and incubated overnight at 4° C. The plates were washed three times with PBS-Tween 20. To prevent non-specific binding, wells were blocked with 100 μl of 8% nonfat dry milk-PBS-Tween 20 and incubated for 1.5 h at 37° C. For IgA analysis, 100 μl of 1:200 dilution of the bile in 8% nonfat dry milk-PBS-Tween 20 was added to the plates in duplicates and incubated for 1.5 h at room temperature. After washing, 100 μl of 1:100,000 dilutions of HRP-labeled anti-chicken IgA (Novus Biologicals, CO, USA) in PBS-Tween 20-5% nonfat dry milk was added to each well and incubated for 1 h at room temperature. The plates were washed with PBS-Tween 20, and the substrate 3,3,5,5-tetramethylbenzidine (TMB) solution (eBioscience, San Diego, Calif., USA) was added to the wells (100 μl/well). The reaction was stopped after 10 min using IN HCl (100 μl/well), and the optical density was read at 450 nm using a microplate ELISA reader. IgA values were reported as the mean optical density.

Effect of Probiotic Supplementation on jejunal histological parameters post Salmonella infection in broiler birds.

The jejunums from 6 samples per treatment group were dehydrated at room temperature in a graded series of alcohols (15 min in 50% ethanol, 15 min in 70% ethanol, 15 min in 95% ethanol, 30 min in 100% ethanol with 1 change at 15 min), cleared in Pro-par (Anatech) for 45 min with 2 changes at 15 and 30 min and infiltrated with paraffin at 60° C. overnight with 1 change at 15 min using a Leica TP 1020 tissue processor (GMI, MN). Paraffin blocks were cross sectioned at 5 μm using a microtome. The sections were mounted on frosted slides (Fisher Scientific) warmed to 37° C. and stained with hematoxylin and eosin. The cross sections were viewed and photographed using an Olympus IX81 microscope and analyzed using CellSens Imaging software (Olympus America) to determine the villi height and crypt depth. Five villi per section and five sections per sample were analyzed.

The experiments were performed and the data processed as one way ANOVA.

Effect of Probiotic Supplementation on Body Weight Gain

No significant treatment effects were observed until 21 d of age.

At 28 d of age, birds challenged with Salmonella had the 11% decreased body weight gain compared to the control groups. Birds that were supplemented with probiotics in the Salmonella challenged groups had only 5.1% decrease in body weight compared to the control group and 5.8% decrease in body weight compared to the groups fed probiotic with no Salmonella infection.

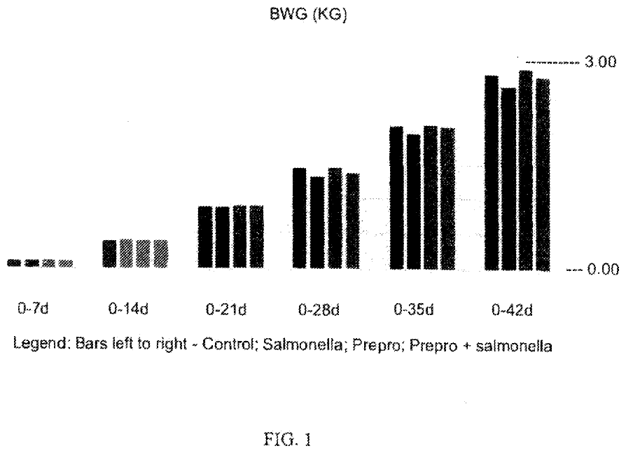

At 42 d f age, birds fed probiotics had numerically higher bodyweight gain of 2.6% compared to the control group. At 42 d of age, birds challenged with Salmonella had the 6% decreased body weight gain compared to the control groups. Birds that were supplemented with probiotics in the Salmonella challenged groups had only 1.2% decrease in body weight compared to the control group and 3.8% decrease in body weight compared to the groups fed probiotic with no Salmonella infection. Birds supplemented with probiotic and challenged with Salmonella had statistically comparable body weight to the control group with no Salmonella. See, FIG. 1.

Effect of Probiotic Supplementation on Feed Consumption

There were no significant treatment effects on feed consumption at most time points.

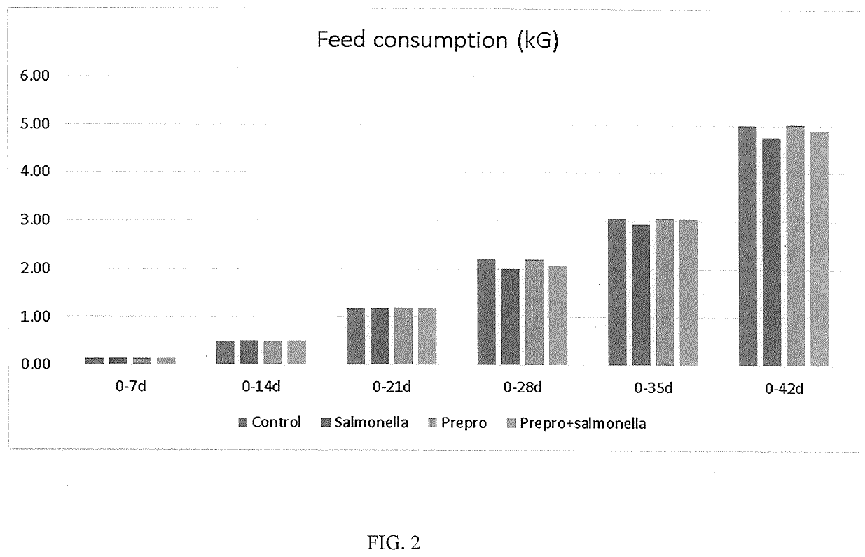

At 28 d of age, birds challenged with Salmonella had the 9.7% decreased feed consumption compared to the control groups. Birds that were supplemented with probiotics in the Salmonella challenged groups had only 6.3% decrease in feed consumption compared to the control group and 5.8% decrease in body weight compared to the groups fed probiotic with no Salmonella infection. Birds supplemented with probiotic and challenged with Salmonella had statistically comparable feed consumption to the control group with no Salmonella as well as prebiotic supplemented groups. See, FIG. 2.

Effect of Probiotic Supplementation on Feed Efficiency

There were no significant treatment effects on feed efficiency at any of the time points studied.

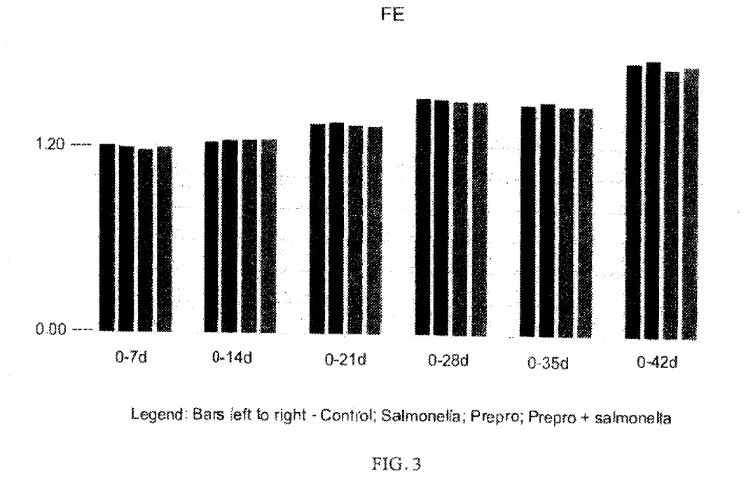

Numerical differences: At 42 d of age, birds fed probiotic had 3 point better feed efficiency compared to the control group. Birds challenged with Salmonella had 6 points poor feed efficiency compared to the control group. Birds fed probiotics and challenged with Salmonella had only 2 point poor efficiency compared to the control group and only 1 point poor feed efficiency compared to the probiotic fed group. See, FIG. 3.

Effect of Probiotic Supplementation on Caecal Salmonella Colonization

There were no detectable levels of Salmonella in the caeca at D0 (preinfection) of Salmonella challenge.

At 5, 12, and 21 d post-Salmonella infection birds in the Salmonella uninfected groups had no detectable levels of Salmonella in the caeca.

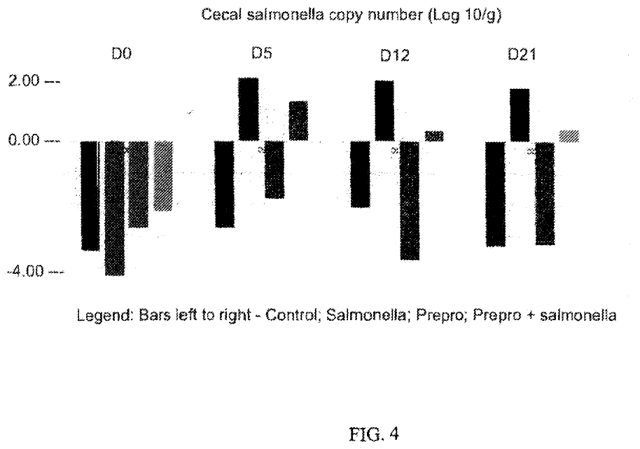

Birds challenged with Salmonella had 1.99, 1.93 and 1.71 log Salmonella CFU/g of caecal contents. Birds supplemented with probiotics and challenged with Salmonella had 0.73, 1.59, and 1.32 log decreased Salmonella CFU/g of caecal contents at 5, 12, and 21 d post-Salmonella infection. See, FIG. 4.

Effect of Probiotic Supplementation on Caecal Campylobacter Colonization

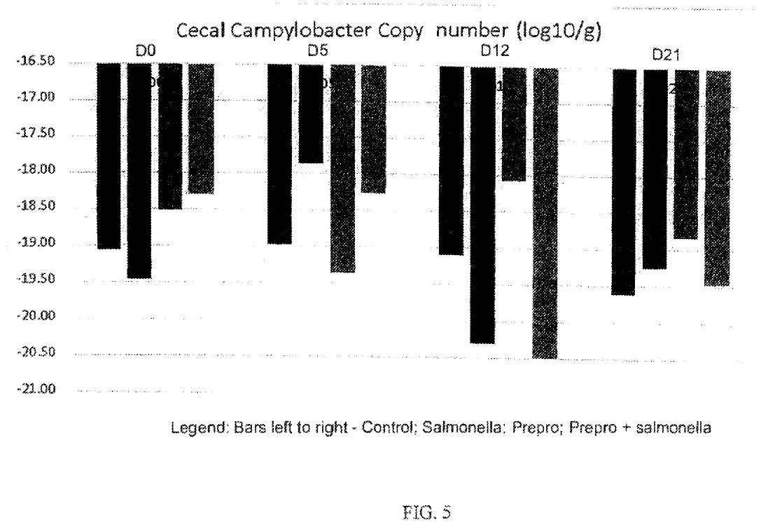

There were no detectable levels of Campylobacter in the caeca at any time point. See, FIG. 5.

Effect of Probiotic Supplementation on Bile Anti-Salmonella Specific IgA

There were no detectable levels of anti-Salmonella IgA in the bile at D0 (preinfection) of the Salmonella challenge.

At 5, 12, and 21 d post-Salmonella infection birds in the Salmonella uninfected groups had no detectable levels of anti-Salmonella IgA in the bile.

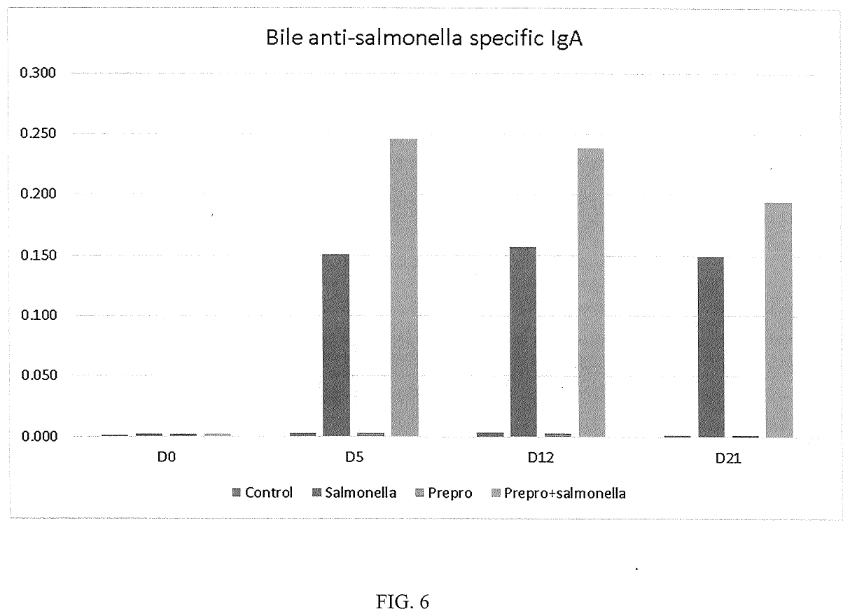

Birds challenged with Salmonella had higher titers of anti-Salmonella IgA in the bile. Birds supplemented with probiotics and challenged with Salmonella had further increased anti-Salmonella titers compared to the birds with Salmonella infection. See, FIG. 6.

Effect of Probiotic Supplementation on serum anti-Salmonella specific IgG

There were no significant treatment effects on serum anti-Salmonella specific IgG at any of the time points studied.

Effect of Probiotic Supplementation on Jejunal Villi Height and Crypt Depth

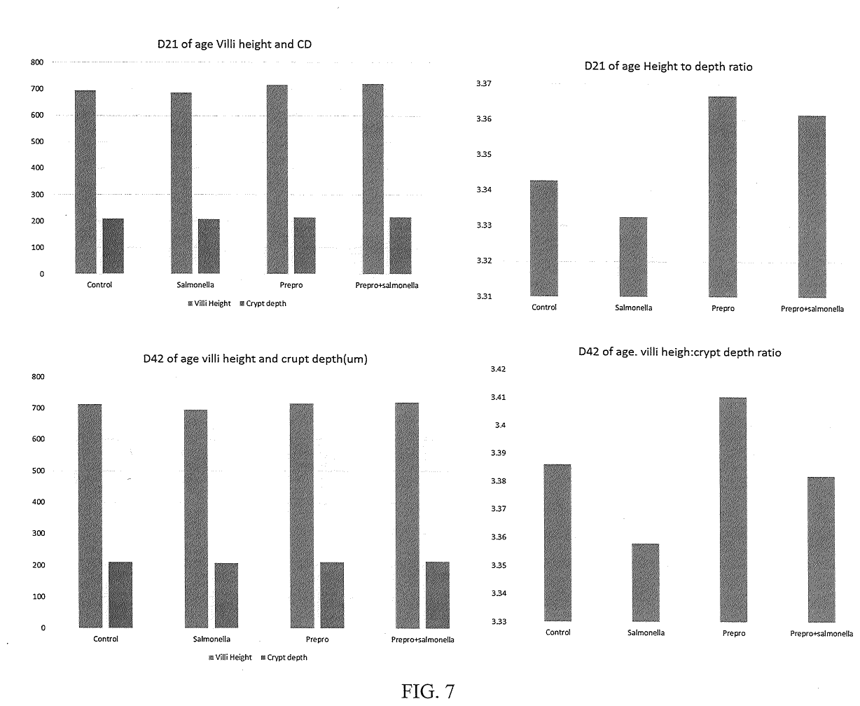

At both 21 and 42 d of age there were significant effects of probiotic supplementation on jejunal villi height. At 21 d of age (pre-Salmonella infection), supplementing probiotics increased the villi height. At 42 d of age (21d post-Salmonella infection), birds challenged with Salmonella had decreased villi height compared to the control groups. Birds supplemented with probiotics had the longest villi height. Birds supplemented with probiotics and challenged with Salmonella had comparable villi height compared to the control group. See, FIG. 7.

Similar to the effects of treatments on villi height effects, at 42 d of age (21d post-Salmonella infection), birds challenged with Salmonella had decreased villi height:crypt depth ratio compared to the control groups. Birds supplemented with probiotics had the highest villi height:crypt depth ratio. Birds supplemented with probiotics and challenged with Salmonella had comparable villi height:crypt depth ratio compared to the control group. See, FIG. 7.

Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entireties for all purposes.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

We claim:

1. A method for treating a chicken for a Salmonella infection, comprising the steps of
administering to the chicken in need of such treatment an effective amount of a spore-based probiotic composition comprising strains Bacillus subtilis (HU58) and Bacillus licheniformis SL-307, each strain comprising Bacillus spores, wherein the Salmonella infection is reduced in severity.

2. The method of claim 1, wherein the probiotic composition comprises a chicken feed including a cellulose carrier.

3. The method of claim 1, wherein the chicken feed comprises Bacillus subtilis (HU58) at a dose of 10 mg/kg feed and Bacillus licheniformis SL-307 at a dose of 100 mg/kg feed.

4. The method of claim 1, wherein a caecal bacterial load of the Salmonella infection is reduced by about 25% copy numbers of S. enteritidis expressed in log 10 CFU/g of caecal contents after about 5 days of treatment.

5. The method of claim 1, wherein a caecal bacterial load of the Salmonella infection is reduced by about 50% copy numbers of S. enteritidis expressed in log 10 CFU/g of caecal contents after about 10 days of treatment.

6. The method of claim 1, wherein the Salmonella infection is derived from Salmonella enterica serovar Enteritidis.

7. A method for treating or preventing a Salmonella infection in a chicken, comprising the steps of
administering to the chicken in need of such treatment an effective amount of a spore-based probiotic composition comprising strains Bacillus subtilis (HU58) and Bacillus licheniformis SL-307, each strain comprising Bacillus spores.

8. The method of claim 7, wherein the probiotic composition comprises a chicken feed including a cellulose carrier.

9. The method of claim 7, wherein the chicken feed comprises Bacillus subtilis (HU58) at a dose of 10 mg/kg feed and Bacillus licheniformis SL-307 at a dose of 100 mg/kg feed.

10. The method of claim 7, wherein a caecal bacterial load of the Salmonella infection is reduced by about 25% copy numbers of S. enteritidis expressed in log 10 CFU/g of caecal contents after about 5 days of treatment.

11. The method of claim 7, wherein a caecal bacterial load of the Salmonella infection is reduced by about 50% copy numbers of S. enteritidis expressed in log 10 CFU/g of caecal contents after about 10 days of treatment.

12. The method of claim 7, wherein the Salmonella infection is derived from Salmonella enterica serovar Enteritidis.


SEQUENCE LISTING (2 sequences)

SEQ ID NO: 1 - DNA, 20 bp, artificial sequence
  synthetic, forward primer
gcagcggttactattgcagc

SEQ ID NO: 2 - DNA, 21 bp, artificial sequence
  synthetic, reverse primer
ctgtgacagggacatttagcg